(19) United States
(12) Patent Application Publication
(10) Pub. No.: US 2012/0095119 A1
(43) Pub. Date: Apr. 19, 2012

(54) PROCESS FOR PRODUCING PURIFIED SYNTHESIS GAS

(76) Inventors: Isaac Cormelis Van Den Born, Amsterdam (NL); Gijsbert Jan Van Heeringen, Amsterdam (NL); Cornelis Jacobus Smit, Amsterdam (NL); Alex Frederik Woldhuis, Amsterdam (NL)

(21) Appl. No.: 13/260,749

(22) PCT Filed: Mar. 30, 2010
(86) PCT No.: PCT/EP2010/054186

§ 371 Date: Jan. 5, 2012

(30) Foreign Application Priority Data
Mar. 30, 2009 (EP) 09156572.1

(51) Int. Cl. C07C 1/04; C07C 29/00; H02K 7/18; C07C 51/00; C01C 1/04; C10L 3/08; C01B 3/50; C07C 41/01

(52) U.S. Cl. 518/704; 252/373; 568/840; 568/671; 562/607; 423/352; 48/197 FM; 290/1 R

(57) ABSTRACT

Disclosed is a process for producing a purified synthesis gas stream from a feed synthesis gas stream. The process includes contacting the feed synthesis gas stream with a water gas shift catalyst in a shift reactor and in the presence of water to obtain a shifted synthesis gas stream enriched in H₂S and in CO₂. H₂S and CO₂ are removed from the shifted synthesis gas stream by contacting the shifted synthesis gas stream with an absorbing liquid to obtain semi-purified synthesis gas and an absorbing liquid rich in H₂S and CO₂. At least part of the absorbing liquid rich in H₂S and CO₂ is heated to obtain heated absorbing liquid rich in H₂S and CO₂ that is then flashed to obtain a flash gas rich in CO₂ and absorbing liquid rich in H₂S. That absorbing liquid rich in H₂S is contacted at elevated temperature with a stripping gas thereby transferring H₂S to the stripping gas to obtain regenerated absorbing liquid and stripping gas rich in H₂S. H₂S in the stripping gas rich in H₂S is converted to elemental sulphur, and H₂S is removed from the semi-purified synthesis gas by converting H₂S in the semi-purified synthesis gas to elemental sulphur to obtain the purified synthesis gas.

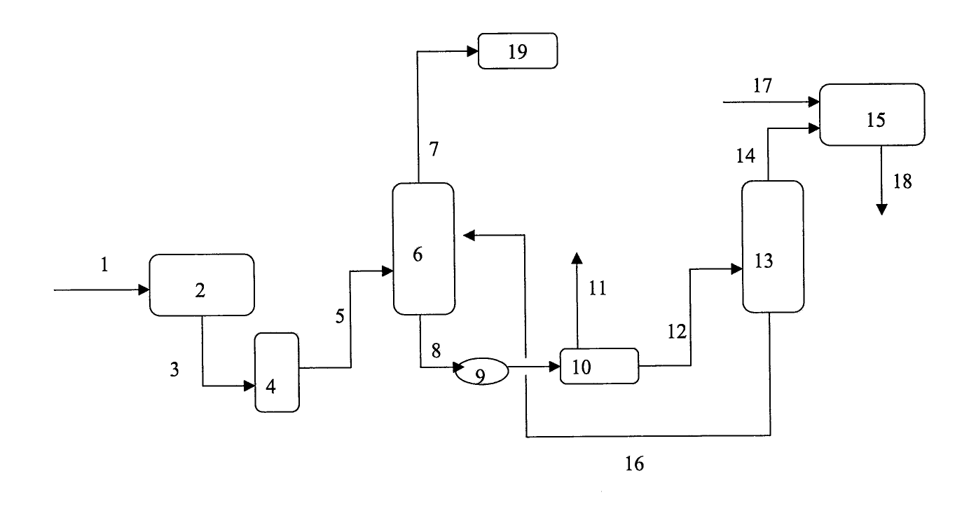

The present invention relates to a process for producing a purified synthesis gas stream from a feed synthesis gas stream comprising contaminants.

Synthesis gas streams are gaseous streams mainly comprising carbon monoxide and hydrogen. Synthesis gas streams are generally produced via partial oxidation or steam reforming of hydrocarbons including natural gas, coal bed methane, distillate oils and residual oil, and by gasification of solid fossil fuels such as biomass or coal or coke.

There are many solid or very heavy (viscous) fossil fuels which may be used as feedstock for generating synthesis gas, including biomass, solid fuels such as anthracite, brown coal, bitumous coal, sub-bitumous coal, lignite, petroleum coke, peat and the like, and heavy residues, e.g. hydrocarbons extracted from tar sands, residues from refineries such as residual oil fractions boiling above 360° C., directly derived from crude oil, or from oil conversion processes such as thermal cracking, catalytic cracking, hydrocracking etc. All such types of fuels have different proportions of carbon and hydrogen, as well as different substances regarded as contaminants.

Depending on the feedstock used to generate synthesis gas, the synthesis gas will contain contaminants such as carbon dioxide, hydrogen sulphide, carbonyl sulphide and carbonyl disulphide while also nitrogen, nitrogen-containing components (e.g. HCN and NH₃), metals, metal carbonyls (especially nickel carbonyl and iron carbonyl), and in some cases mercaptans.

Purified synthesis gas can be used in catalytical chemical conversions or to generate power. A substantial portion of the world's energy supply is provided by combustion of fuels, especially natural gas or synthesis gas, in a power plant. Synthesis gas is combusted with air in one or more gas turbines and the resulting gas is used to produce steam. The steam is then used to generate power.

An especially suitable system for using synthesis gas in power generation is the Integrated Gasification Combined Cycle (IGCC) system. IGCC systems were devised as a way to use coal as the source of fuel in a gas turbine plant. IGCC is a combination of two systems. The first system is coal gasification, which uses coal to create synthesis gas. The syngas is then purified to remove contaminants. The purified synthesis gas may be used in the combustion turbine to produce electricity.

The second system in IGCC is a combined-cycle, or power cycle, which is an efficient method of producing electricity commercially. A combined cycle includes a combustion turbine/generator, a heat recovery steam generator (HRSG), and a steam turbine/generator. The exhaust heat from the combustion turbine may be recovered in the HRSG to produce steam. This steam then passes through a steam turbine to power another generator, which produces more electricity. A combined cycle is generally more efficient than conventional power generating systems because it re-uses waste heat to produce more electricity. IGCC systems are clean and generally more efficient than conventional coal plants.

As set out hereinabove, when synthesis gas is used for power production, removal of contaminants is often required to avoid deposition of contaminants onto the gas turbine parts.

When synthesis gas is used in catalytical chemical conversions, removal of contaminants to low levels is required to prevent catalyst poisoning.

Processes for producing a purified synthesis gas stream generally involve the use of expensive line-ups. For example, cold methanol may be used to remove hydrogen sulphide and carbon dioxide by physical absorption. The concentrations of these contaminants in the purified synthesis gas will still be relatively high. For applications where the synthesis gas is to be catalytically converted, lower contaminant concentrations would be required. Purifying the synthesis gas streams to a higher degree using a methanol-based process would be uneconomical due to the disproportionately large amounts of energy required to cool and later to regenerate the methanol.

It is an object of the present invention to provide an optimised process for purification of a synthesis gas stream derived from a range of carbonaceous fuels, such that the purified synthesis gas is suitable for further use, especially for power production.

To this end, the invention provides a process for producing a purified synthesis gas stream from a feed synthesis gas stream comprising besides the main constituents carbon monoxide and hydrogen also hydrogen sulphide, carbonyl sulphide and/or hydrogen cyanide and optionally ammonia, the process comprising the steps of: (a) contacting the feed synthesis gas stream with a water gas shift catalyst in a shift reactor in the presence of water and/or steam to react at least part of the carbon monoxide to carbon dioxide and hydrogen and at least part of the hydrogen cyanide to ammonia and/or at least part of the carbonyl sulphide to hydrogen sulphide, to obtain a shifted synthesis gas stream enriched in H₂S and in CO₂ and optionally comprising ammonia; (b) removing H₂S and CO₂ from the shifted synthesis gas stream by contacting the shifted synthesis gas stream with an absorbing liquid to obtain semi-purified synthesis gas and an absorbing liquid rich in H₂S and CO₂; (c) heating at least part of the absorbing liquid rich in H₂S and CO₂ in a heater to obtain heated absorbing liquid rich in H₂S and CO₂; (d) de-pressurising the heated absorbing liquid rich in H₂S and CO₂ in a flash vessel, thereby obtaining flash gas rich in CO₂ and absorbing liquid rich in H₂S; (e) contacting the absorbing liquid rich in H₂S at elevated temperature with a stripping gas, thereby transferring H₂S to the stripping gas to obtain regenerated absorbing liquid and stripping gas rich in H₂S; (f) converting H₂S in stripping gas rich in H₂S to elemental sulphur; (g) removing H₂S from the semi-purified synthesis gas by converting H₂S in the semi-purified synthesis gas to elemental sulphur to obtain the purified synthesis gas.

The process enables producing a purified synthesis gas having low levels of contaminants, suitably in the ppmv or even in the ppbv range. The purified synthesis gas, because of its low level of contaminants, especially with regard to HCN and/or COS, is suitable for many uses, especially for use as feedstock to generate power or for use in a catalytic chemical reaction. The purified synthesis gas is especially suitable for use in an Integrated Gasification Combined Cycle (IGCC).

An important advantage of the process is that in step (d), a CO₂ rich stream is obtained at a relatively high pressure suitably in the range of from 5 to 10 bara. This facilitates the use of the CO₂-rich stream for enhanced oil recovery or for reinjection into a subterranean formation or aquifer, with less equipment needed for further compression of the CO₂-rich stream.

Another advantage of the process is that in step (e) a stripping gas rich in H₂S and comprising little CO₂ is obtained, even when processing a feed synthesis gas stream comprising substantial amounts of CO₂. Suitably, the H₂S concentration in stripping gas rich in H₂S will be more than 30 volume %. Such a stripping gas is a suitable feed for a sulphur recovery unit, where H₂S is converted to elemental sulphur. A high concentration of H₂S in the feed to a sulphur recovery unit enables the use of a smaller sulphur recovery unit and thus a lower capital and operational expenditure.

Typically, the feed synthesis gas is generated from a feedstock in a synthesis generation unit such as a high temperature reformer, an autothermal reformer or a gasifier. See for example Maarten van der Burgt et al., in “The Shell Middle Distillate Synthesis Process, Petroleum Review Apr. 1990 pp. 204-209”.

Apart from coal and heavy oil residues, there are many solid or very heavy (viscous) fossil fuels which may be used as feedstock for generating synthesis gas, including solid fuels such as anthracite, brown coal, bitumous coal, sub-bitumous coal, lignite, petroleum coke, peat and the like, and heavy residues, e.g. hydrocarbons extracted from tar sands, residues from refineries such as residual oil fractions boiling above 360° C., directly derived from crude oil, or from oil conversion processes such as thermal cracking, catalytic cracking, hydrocracking etc. All such types of fuels have different proportions of carbon and hydrogen, as well as different substances regarded as contaminants.

Synthesis gas generated in reformers usually comprises besides the main constituents carbon monoxide and hydrogen, also carbon dioxide, steam, various inert compounds and impurities such as HCN and sulphur compounds. Synthesis gas generated in gasifiers conventionally comprises lower levels of carbon dioxide.

The synthesis gas exiting a synthesis gas generation unit may comprise particulate matter, for example soot particles. Preferably, these soot particles are removed, for example by contacting the synthesis gas exiting a synthesis gas generation unit with scrubbing liquid in a soot scrubber to remove particulate matter, in particular soot, thereby obtaining the feed synthesis gas comprising besides the main constituents CO and H₂ also H₂S and optionally CO₂, HCN and/or COS.

Suitably, the amount of H₂S in the feed synthesis gas will be in the range of from 1 ppmv to 20 volume %, typically from 1 ppmv to 10 volume %, based on the synthesis gas.

If applicable, the amount of CO₂ in the feed synthesis gas is from about 0.5 to 10 vol %, preferably from about 1 to 10 vol %, based on the synthesis gas.

If HCN is present, the amount of HCN in the feed synthesis gas will generally be the range of from about 1 ppbv to about 500 ppmv.

If COS is present, the amount of COS in the feed synthesis gas will generally be in the range of from about 1 ppbv to about 100 ppmv.

In step (a), the feed synthesis gas stream is contacted with a water gas shift catalyst to react at least part of the carbon monoxide with water. The water shift conversion reaction is well known in the art. Generally, water, usually in the form of steam, is mixed with the feed synthesis gas stream to form carbon dioxide and hydrogen. The catalyst used can be any of the known catalysts for such a reaction, including iron, chromium, copper and zinc. Copper on zinc oxide is an especially suitable shift catalyst.

In a preferred embodiment of step (a), carbon monoxide in the feed synthesis gas stream is converted with a low amount of steam in the presence of a catalyst as present in one or more fixed bed reactors. A series of shift reactors may be used wherein in each reactor a water gas shift conversion step is performed. The content of carbon monoxide, on a dry basis, in the feed synthesis gas stream as supplied to the first or only water gas shift reactor is preferably at least 50 vol. %, more preferably between 55 and 70 vol. %. The feed synthesis gas stream preferably contains hydrogen sulphide in order to keep the catalyst sulphided and active. The minimum content of hydrogen sulphide will depend on the operating temperature of the shift reactor, on the space velocity (GHSV) and on the sulphur species present in the feed synthesis gas stream. Preferably at least 300 ppm H₂S is present in the feed synthesis gas stream. There is no limitation on the maximum amount of H₂S from a catalyst activity point of view.

In the preferred embodiment of step (a), the steam to carbon monoxide molar ratio in the feed synthesis gas stream as it enters the first or only water gas shift reactor is preferably between 0.2:1 and 0.9:1. The temperature of the feed synthesis gas stream as it enters the shift reactor is preferably between 190 and 230° C. In addition it is preferred that the inlet temperature is between 10 and 60° C. above the dewpoint of the feed to each water gas shift conversion step. The space velocity in the reactor is preferably between 6000-9000 h⁻¹. The pressure is preferably between 2 and 5 MPa and more preferably between 3 and 4.5 MPa.

The conversion of carbon monoxide may generally not be 100% because of the sub-stoichiometric amount of steam present in the feed of the reactor. In a preferred embodiment the content of carbon monoxide in the shift reactor effluent, using a fixed bed reactor, will be between 35 and 50 vol. % on a dry basis, when starting from a feed synthesis gas stream comprising between 55 and 70 vol. % carbon monoxide, on a dry basis, and a steam/CO ratio of 0.2 to 0.3 molar. If a further conversion of carbon monoxide is desired it is preferred to subject the shift reactor effluent to a next water gas shift conversion step.

The preferred steam/water to carbon monoxide molar ratio, inlet temperature and space velocity for such subsequent water gas shift conversion steps is as described for the first water gas shift conversion step. As described above the feed synthesis gas stream is suitably obtained from a gasification process and is suitably subjected to a water scrubbing step. In such a step water will evaporate and end up in the syngas mixture. The resultant steam to CO molar ratio in such a scrubbed syngas will suitably be within the preferred ranges as described above. This will result in that no steam or water needs to be added to the syngas as it is fed to the first water gas shift conversion step. In order to achieve the desired steam to CO molar ranges for the subsequent steps steam or boiler feed water will have to be added to the effluent of each previous step.

The water gas shift step may be repeated to stepwise lower the carbon monoxide content in the shift reactor effluent of each next shift reactor to a CO content, on a dry basis, of below 5 vol. %. It has been found that in 4 to 5 steps, or said otherwise, in 4 to 5 reactors such a CO conversion can be achieved.

It has been found that it is important to control the temperature rise in each shift reactor. It is preferred to operate each shift reactor such that the maximum temperature in the catalyst bed in a single reactor does not exceed 440° C. and more preferably does not exceed 400° C. At higher temperatures the exothermal methanation reaction can take place, resulting in an uncontrolled temperature rise.

The catalyst used in the shift reactor is preferably a water gas shift catalyst, which is active at the preferred low steam to CO molar ratio and active at the relatively low inlet temperature without favouring side reactions such as methanation. Suitably the catalyst comprises a carrier and the oxides or sulphides of molybdenum (Mo), more preferably a mixture of the oxides or sulphides of molybdenum (Mo) and cobalt (Co) and even more preferably also comprising copper (Cu) tungsten (W) and/or nickel (Ni). The catalyst suitably also comprises one or more promoters/inhibitors such as potassium (K), lanthanum (La), manganese (Mn), cerium (Ce) and/or zirconium (Zr). The carrier may be a refractory material such as for example alumina, MgAl₂O₄ or MgO—Al₂O₃—TiO₂.

An example of a suitable catalyst comprises an active γ-Al₂O₃ carrier and between 1-8 wt % CoO and between 6-10 wt % MoO₃. The catalyst is preferably present as an extrudate.

In a preferred embodiment of step (a), the feed synthesis gas stream comprises at least 50 vol. % of carbon monoxide, and the steam to carbon monoxide molar ratio in the feed synthesis gas stream as it enters the shift reactor or reactors is preferably between 0.2:1 and 0.9:1 and the temperature of the feed synthesis gas stream as it enters the shift reactor or reactors is between 190 and 230° C.

Additional reactions taking place in step (a) are the conversion of HCN to ammonia and/or the conversion of COS to H₂S. Thus, the shifted gas stream obtained in step (a) will be depleted in HCN and/or in COS.

Optionally, the shifted gas stream obtained in step (a) is cooled to remove water and if applicable, ammonia. Preferably, at least 50%, more preferably at least 80% and most preferably at least 90% of the water and if applicable ammonia is removed, based on the shifted gas stream.

In step (b), the shifted synthesis gas is contacted with absorbing liquid in an absorber to remove H₂S and CO₂, thereby obtaining semi-purified synthesis gas and absorbing liquid rich in H₂S and CO₂.

Suitable absorbing liquids may comprise physical solvents and/or chemical solvents. Physical solvents are understood to be solvents that show little or no chemical interaction with H₂S and/or CO₂. Suitable physical solvents include sulfolane(cyclo-tetramethylenesulfone and its derivatives), aliphatic acid amides, N-methyl-pyrrolidone, N-alkylated pyrrolidones and the corresponding piperidones, methanol, ethanol and mixtures of dialkylethers of polyethylene glycols. Chemical solvents are understood to be solvents that can show chemical interaction with H₂S and/or CO₂. Suitable chemical solvents include amine type solvents, for example primary, secondary and/or tertiary amines, especially amines that are derived of ethanolamine, especially monoethanol amine (MEA), diethanolamine (DEA), triethanolamine (TEA), diisopropanolamine (DIPA) and methyldiethanolamine (MDEA) or mixtures thereof.

A preferred absorbing liquid comprises a physical and a chemical solvent.

An advantage of using absorption liquids comprising both a chemical and a physical solvent is that they show good absorption capacity and good selectivity for H₂S and/or CO₂ against moderate investment costs and operational costs.

An especially preferred absorbing liquid comprises a secondary or tertiary amine, preferably an amine compound derived from ethanol amine, more especially DIPA, DEA, MMEA (monomethyl-ethanolamine), MDEA, or DEMEA (diethyl-monoethanolamine), preferably DIPA or MDEA.

Step (b) is preferably performed at a temperature in the range of from 15 to 90° C., more preferably at a temperature of at least 20° C., still more preferably from 25 to 80° C., even more preferably from 40 to 65° C., and most preferably at about 55° C. At the preferred temperatures, better removal of H₂S and CO₂ is achieved. Step (b) is suitably carried out at a pressure in the range of from 15 to 90 bara, preferably from 20 to 80 bara, more preferably from 30 to 70 bara.

Step (b) is suitably carried out in an absorber having from 5-80 contacting layers, such as valve trays, bubble cap trays, baffles and the like. Structured packing may also be applied. A suitable solvent/feed gas ratio is from 1.0 to 10 (w/w), preferably between 2 and 6 (w/w).

In step (c), at least part of the absorbing liquid rich in H₂S and CO₂ is heated. Suitably, the absorbing liquid rich in H₂S and CO₂ is heated to a temperature in the range of from 90 to 120° C.

In step (d), the heated absorbing liquid is de-pressurised in a flash vessel, thereby obtaining flash gas enriched in CO₂ and absorbing liquid enriched in H₂S. Step (d) is carried out at a lower pressure compared to the pressure in step (b), but preferably at a pressure above atmospheric pressure. Suitably, the de-pressurising is done such that as much CO₂ as possible is released from the heated absorbing liquid. Preferably, step (d) is carried out at a pressure in the range of from 2 to 10 bara, more preferably from 5 bara to 10 bara. It has been found that at these preferred pressures, a large part of the CO₂ is separated from the absorbing liquid rich in H₂S and CO₂, resulting in flash gas rich in CO₂.

Suitably, in step (d) at least 50%, preferably at least 70% and more preferably at least 80% of the CO₂ is separated from the absorbing liquid rich in H₂S and CO₂. Step (d) results in flash gas rich in CO₂ and absorbing liquid rich in H₂S. Preferably, the flash gas obtained in step (d) comprises in the range of from 10 to 100 volume %, preferably from 50 to 100% of CO₂.

The flash gas rich in CO₂ is suitable for further uses. In applications where the CO₂-rich gas needs to be at a high pressure, for example when it will be used for injection into a subterranean formation, it is an advantage that the CO₂-rich flash gas is already at an elevated pressure as this reduces the equipment and energy requirements needed for further pressurisation.

In a preferred embodiment, the flash gas rich in CO₂ is used for enhanced oil recovery, suitably by injecting it into an oil reservoir where it tends to dissolve into the oil in place, thereby reducing its viscosity and thus making it more mobile for movement towards the producing well.

In another embodiment, the CO₂-rich gas stream is further pressurised and pumped into an aquifer or an empty oil reservoir for storage.

For all the above options, the flash gas rich in CO₂ needs to be compressed. Suitably, the flash gas rich in CO₂ is compressed to a pressure in the range of from 60 to 300 bara, more preferably from 80 to 300 bara. Normally, a series of compressors would be needed to pressurise the CO₂-enriched gas stream to the desired high pressures. Pressurising a CO₂-rich gas stream from atmospheric pressure to a pressure of about 10 bara requires a large and expensive compressor. As the process produces a CO₂-rich gas already at elevated pressure, savings on the compressor equipment can be realised.

In step (e), the absorbing liquid comprising H₂S is contacted at elevated temperature with a stripping gas, thereby transferring H₂S to the stripping gas to obtain regenerated absorbing liquid and stripping gas rich in H₂S. Step (e) is suitably carried out in a regenerator. Preferably, the elevated temperature in step (e) is a temperature in the range of from 70 to 150° C. The heating is preferably carried out with steam or hot oil. Preferably, the temperature increase is done in a stepwise mode. Suitably, step (e) is carried out at a pressure in the range of from 1 to 3 bara, preferably from 1 to 2.5 bara.

In step (f), hydrogen sulphide is reacted with sulphur dioxide in the presence of a catalyst to form elemental sulphur. This reaction is known in the art as the Claus reaction. Preferably, the stripping gas rich in H₂S and a gas stream comprising SO₂ are supplied to a sulphur recovery system comprising one or more Claus catalytic stages in series. Each of the Claus catalytic stages comprises a Claus catalytic reactor coupled to a sulphur condenser. In the Claus catalytic reactor, the Claus reaction between H₂S and SO₂ to form elemental sulphur takes place. A product gas effluent comprising elemental sulphur as well as unreacted H₂S and/or SO₂ exits the Claus catalytic reactor and is cooled below the sulphur dew point in the sulphur condenser coupled to the Claus catalytic reactor to condense and separate most of the elemental sulphur from the Claus reactor effluent. The reaction between H₂S and SO₂ to form elemental sulphur is exothermic, normally causing a temperature rise across the Claus catalytic reactor with an increasing concentration of H₂S in the incoming stripping gas rich in H₂S. At an H₂S concentration in the stripping gas rich in H₂S above 30% or even above 15%, it is believed that the heat generated in the Claus catalytic reactors will be such that the temperature in the Claus reactors will exceed the desired operating range if sufficient SO₂ is present to react according to the Claus reaction. Preferably, the operating temperature of the Claus catalytic reactor is maintained in the range of from about 200 to about 500° C., more preferably from about 250 to 350° C.

Step (b) results in semi-purified synthesis gas and absorbing liquid rich in H₂S and CO₂.

The semi-purified synthesis gas obtained in step (b) comprises predominantly hydrogen and carbon monoxide and CO₂ and low levels of H₂S and optionally other contaminants.

In step (g), at least part of the hydrogen sulphide in the semi-purified synthesis gas is converted to elemental sulphur.

In one embodiment of step (g), hydrogen sulphide is converted to elemental sulphur by contacting the semi-purified synthesis gas with an aqueous reactant solution containing solubilized Fe(III) chelate of an organic acid, at a temperature below the melting point of sulphur, and at a sufficient solution to gas ratio and conditions effective to convert H₂S to elemental sulphur and inhibit sulphur deposition, thereby producing a gas-solution mixture comprising sour gas and aqueous reactant solution containing dispersed sulphur particles.

The iron chelates employed are coordination complexes in which irons forms chelates with an acid. The acid may have the formula

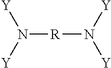

wherein
from two to four of the groups Y are selected from acetic and propionic acid groups;
from zero to two of the groups Y are selected from 2-hydroxy-ethyl, 2-hydroxypropyl, and

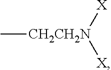

wherein X is selected from acetic and propionic acid groups; and
R is ethylene, propylene or isopropylene or alternatively cyclo-hexane or benzene where the two hydrogen atoms replaced by nitrogen are in the 1,2 position, and mixtures thereof.

Exemplary chelating agents for the iron include aminoacetic acids derived from ethylenediamine, diethylenetriamine, 1,2-propylenediamine, and 1,3-propylenediamine, such as EDTA (ethylenediamine tetraacetic acid), HEEDTA (N-2-hydroxyethyl ethylenediamine triacetic acid), DETPA (diethylenetriamine pentaacetic acid); aminoacetic acid derivatives of cyclic, 1,2-diamines, such as 1,2-di-amino cyclohexane-N,N-tetraacetic acid, and 1,2-phenylene-diamine-N,N-tetraacetic acid, and the amides of polyamino acetic acids disclosed in Bersworth U.S. Pat. No. 3,580,950. Suitably, the ferric chelate of N-(2-hydroxyethyl)ethylenediamine triacetic acid (HEEDTA) is used.

A further suitable iron chelate is the coordination complex in which iron forms a chelate with nitrilotriacetic acid (NTA).

The iron chelates are supplied in solution as solubilized species, such as the ammonium or alkali metal salts (or mixtures thereof) of the iron chelates. As used herein, the term “solubilized” refers to the dissolved iron chelate or chelates, whether as a salt or salts of the aforementioned cation or cations, or in some other form, in which the iron chelate or chelates exist in solution. Where solubility of the chelate is difficult, and higher concentrations of chelates are desired, the ammonium salt may be utilized, as described in European patent application publication No. 215,505.

However, the invention may also be employed with more dilute solutions of the iron chelates, wherein the steps taken to prevent iron chelate precipitation are not critical.

Regeneration of the reactant is preferably accomplished by the utilization of oxygen, preferably as air. As used herein, the term “oxygen” is not limited to “pure” oxygen, but includes air, air enriched with oxygen, or other oxygen-containing gases. The oxygen will accomplish two functions, the oxidation of Fe(II) iron of the reactant to the Fe(III) state, and the stripping of any residual dissolved gas (if originally present) from the aqueous admixture. The oxygen (in whatever form supplied) is supplied in a stoichiometric equivalent or excess with respect to the amount of solubilized iron chelate to be oxidized to the Fe(III) state. Preferably, the oxygen is supplied in an amount of from about 20 percent to about 500 percent excess. Electrochemical regeneration may also be employed.

Step (g) results in purified synthesis gas. The amount of H₂S in the purified synthesis gas is preferably 1 ppmv or less, more preferably 100 ppbv or less, still more preferably 10 ppbv or less and most preferably 5 ppbv or less, based on the purified synthesis gas.

The purified synthesis gas obtainable by the process is suitable for many uses, including generation of power or conversion in chemical processes. Thus, the invention also includes purified synthesis gas, obtainable by the process.

In a preferred embosiment, the purified synthesis gas is used in catalytic processes, preferably selected from the group of Fischer-Tropsch synthesis, methanol synthesis, di-methyl ether synthesis, acetic acid synthesis, ammonia synthesis, methanation to make substitute natural gas (SNG) and processes involving carbonylation or hydroformylation reactions.

In another preferred embodiment, the purified synthesis gas is used for power generation, especially in an IGCC system.

In the IGCC system, typically, fuel and an oxygen-containing gas are introduced into a combustion section of a gas turbine. In the combustion section of the gas turbine, the fuel is combusted to generate a hot combustion gas. The hot combustion gas is expanded in the gas turbine, usually via a sequence of expander blades arranged in rows, and used to generate power via a generator. Suitable fuels to be combusted in the gas turbine include natural gas and synthesis gas.

Hot exhaust gas exiting the gas turbine is introduced into to a heat recovery steam generator unit, where heat contained in the hot exhaust gas is used to produce a first amount of steam.

Suitably, the hot exhaust gas has a temperature in the range of from 350 to 700° C., more preferably from 400 to 650° C. The composition of the hot exhaust gas can vary, depending on the fuel gas combusted in the gas turbine and on the conditions in the gas turbine.

The heat recovery steam generator unit is any unit providing means for recovering heat from the hot exhaust gas and converting this heat to steam. For example, the heat recovery steam generator unit can comprise a plurality of tubes mounted stackwise. Water is pumped and circulated through the tubes and can be held under high pressure at high temperatures. The hot exhaust gas heats up the tubes and is used to produce steam.

The heat recovery steam generator unit can be designed to produce three types of steam: high pressure steam, intermediate pressure steam and low pressure steam.

Preferably, the steam generator is designed to produce at least a certain amount of high pressure steam, because high pressure steam can be used to generate power. Suitably, high-pressure steam has a pressure in the range of from 90 to 150 bara, preferably from 90 to 125 bara, more preferably from 100 to 115 bara. Suitably, low-pressure steam is also produced, the low-pressure steam preferably having a pressure in the range of from 2 to 10 bara, more preferably from to 8 bara, still more preferably from 4 to 6 bara.

In the heat recovery steam generator unit preferably high pressure steam is produced in a steam turbine, which high pressure steam is converted to power, for example via a generator coupled to the steam turbine.

In an especially preferred embodiment, a portion of the shifted synthesis gas stream, optionally after removal of contaminants, is used for hydrogen manufacture, such as in a Pressure Swing Adsorption (PSA) step. The proportion of the shifted synthesis gas stream used for hydrogen manufacture will generally be less than 15% by volume, preferably approximately 1-10% by volume. The hydrogen manufactured in this way can then be used as the hydrogen source in hydrocracking of the products of the hydrocarbon synthesis reaction. This arrangement reduces or even eliminates the need for a separate source of hydrogen, e.g. from an external supply, which is otherwise commonly used where available. Thus, the carbonaceous fuel feedstock is able to provide a further reactant required in the overall process of biomass or coal to liquid products conversion, increasing the self-sufficiency of the overall process.

The invention will now be illustrated using the following non-limiting embodiment with reference to the schematic FIGURE.

In the FIGURE, synthesis gas comprising besides the main constituents of CO and H₂ also H₂S, HCN and COS is led via line 1 to shift reactor 2, where CO is catalytically converted to CO₂ in the presence of water. Also, conversion of HCN and COS to respectively NH₃ and H₂S takes place. The resulting shifted synthesis gas, depleted in HCN and in COS, is optionally washed in scrubber 4 to remove any NH₃ formed and led via line 5 to absorber 6. In absorber 6, the synthesis gas depleted in HCN and in COS is contacted with absorbing liquid, thereby transferring H₂S and CO₂ from the synthesis gas to the absorbing liquid to obtain absorbing liquid rich in H₂S and CO₂ and semi-purified synthesis gas. The semi-purified synthesis gas leaves absorber 6 via line 7. The absorbing liquid rich in H₂S and CO₂ is led via line 8 to heater 9, where it is heated. The resulting heated absorbing liquid is de-pressurised in flash vessel 10, thereby obtaining flash gas rich in CO₂ and absorbing liquid rich in H₂S. The flash gas rich in CO₂ is led from vessel 10 via line 11 to be used elsewhere. The absorbing liquid rich in H₂S is led via line 12 to regenerator 13, where it is contacting at elevated temperature with a stripping gas, thereby transferring H₂S to the stripping gas to obtain regenerated absorbing liquid and stripping gas rich in H₂S. The stripping gas rich in H₂S is led from regenerator 13 via line 14 to Claus reactor 15. Regenerated absorbing liquid is led back to absorber 6 via line 16. SO₂ is supplied to the Claus reactor via line 17. In the Claus reactor, catalytic conversion of H₂S and SO₂ to elemental sulphur takes place. The elemental sulphur is led from the Claus reactor via line 18. Semi-purified synthesis gas is led from absorber 6 via line 7 to a polishing unit 19, where remaining H₂S is converted to elemental sulphur.

1. A process for producing a purified synthesis gas stream from a feed synthesis gas stream, comprising besides the main constituents carbon monoxide and hydrogen, also hydrogen sulphide, carbonyl sulphide and/or hydrogen cyanide and optionally ammonia, the process comprising the steps of:
(a) contacting the feed synthesis gas stream with a water gas shift catalyst in a shift reactor in the presence of water and/or steam to react at least part of the carbon monoxide to carbon dioxide and hydrogen and at least part of the hydrogen cyanide to ammonia and/or at least part of the carbonyl sulphide to hydrogen sulphide, to obtain a shifted synthesis gas stream enriched in H₂S and in CO₂ and optionally comprising ammonia;
(b) removing H₂S and CO₂ from the shifted synthesis gas stream by contacting the shifted synthesis gas stream with an absorbing liquid to obtain a semi-purified synthesis gas and an absorbing liquid rich in H₂S and CO₂;
(c) heating at least part of the absorbing liquid rich in H₂S and CO₂ in a heater to obtain heated absorbing liquid rich in H₂S and CO₂;
(d) de-pressurising the heated absorbing liquid rich in H₂S and CO₂ in a flash vessel, thereby obtaining flash gas rich in CO₂ and absorbing liquid rich in H₂S;
(e) contacting the absorbing liquid rich in H₂S at elevated temperature with a stripping gas, thereby transferring H₂S to the stripping gas to obtain regenerated absorbing liquid and stripping gas rich in H₂S;
(f) converting H₂S in stripping gas rich in H₂S to elemental sulphur; and
(g) removing H₂S from the semi-purified synthesis gas by converting H₂S in the semi-purified synthesis gas to elemental sulphur to obtain the purified synthesis gas.

2. A process according to claim 1, wherein the shifted synthesis gas stream enriched in H₂S and in CO₂ and optionally comprising ammonia obtained in step (a) is cooled to remove water and optionally ammonia.

3. A process according to claim 1, wherein the water/steam to carbon monoxide molar ratio in the feed synthesis gas stream as it enters the shift reactor is in the range of from 0.2:1 to 0.9:1 and wherein the temperature of the feed synthesis gas stream as it enters the shift reactor is in the range of from 190 to 230° C. and wherein the feed synthesis gas stream comprises at least 50 volume % of carbon monoxide, on a dry basis.

4. A process according to claim 1, wherein in step (f) H₂S is reacted with sulphur dioxide in the presence of a catalyst that is a non-promoted spherical activated alumina or titania, to form elemental sulphur.

5. A process according to claim 4, wherein the stripping gas rich in H₂S comprises in the range of from 30 to 90 volume of H₂S.

6. A process according to claim 1, wherein step (c) is performed at a temperature in the range of from 90 to 120° C.

7. A process according to claim 1, wherein step (d) is performed at a pressure in the range of from 2 to 10 bara.

8. A process according to claim 1, wherein the flash gas obtained in step (d) comprises in the range of from 10 to 100 volume % of CO₂.

9. A process according to claim 1, wherein step (g) comprises contacting the semi -purified synthesis gas stream with an aqueous reactant solution containing solubilized Fe(III) chelate of an organic acid, at a temperature below the melting point of sulphur, and at a sufficient solution to gas ratio and conditions effective to convert H₂S to sulphur and inhibit sulphur deposition, thereby producing a gas-solution mixture comprising sour gas and aqueous reactant solution.

10. A process according to claim 1, wherein step (g) comprises reacting H₂S with sulphur dioxide in the presence of a catalyst to form elemental sulphur.

11. A process according to claim 10, wherein the catalyst is non-promoted spherical activated alumina or titania.

12. A process according to, wherein step (b) is performed at a temperature in the range of from 10 to 80° C.

13. A process according to claim 1, wherein step (e) is performed at elevated pressure in the range of from 1.5 to 50 bara.

14. A process according to claim 1, wherein the flash gas rich in CO₂ gas stream is compressed to a pressure in the range of from 60 to 300 bara and injected into a subterranean formation for use in enhanced oil recovery or for storage into an aquifer reservoir or for storage into an empty oil reservoir.

15. A process according to claim 1, wherein the purified synthesis gas is used in a combustion turbine to produce electricity.

16. A process according to claim 1, wherein the purified synthesis gas is used in catalytic processes selected from the group consisting of Fischer-Tropsch synthesis, methanol synthesis, di-methyl ether synthesis, acetic acid synthesis, ammonia synthesis, methanation to make substitute natural gas (SNG) and processes involving carbonylation or hydroformylation reactions.
